(19) United States
(12) Patent Application Publication
(10) Pub. No.: US 2011/0244058 A1
(43) Pub. Date: Oct. 6, 2011

(54) Compositions and methods for treatment of pulmonary diseases and conditions

(76) Inventor: Gerald Horn, Deerfield, IL (US)

(21) Appl. No.: 13/066,368

(22) Filed: Apr. 13, 2011

Related U.S. Application Data
(63) Continuation-in-part of application No. 12/798,925, filed on Apr. 14, 2010.
Application No. 12/798,925 is a continuation-in-part of application No. 12/460,970, filed on Jul. 27, 2009.
(60) Provisional application No. 61/287,518, filed on Dec. 17, 2009.
(60) Provisional application No. 61/137,714, filed on Aug. 1, 2008.
(60) Provisional application No. 61/192,777, filed on Sep. 22, 2008.
(60) Provisional application No. 61/203,120, filed on Dec. 18, 2008.
(60) Provisional application No. 61/207,481, filed on Feb. 12, 2009.

(51) Int. Cl. A61K 33/14; C07D 233/56; A61K 31/4164; C07C 211/35; A61K 31/135; C07D 403/12; A61K 31/498; A61K 33/06; A61P 11/00; A61P 11/06; A61P 31/12

(52) U.S. Cl. 424/681; 548/346.1; 514/396; 564/305; 514/646; 544/353; 514/249; 424/682

(57) ABSTRACT

The invention provides compositions and methods for treating pulmonary diseases and conditions. The provided compositions and methods utilize either low concentrations of selective α-2 adrenergic receptor agonists having a binding affinity of 300 fold or greater for α-2 over α-1 adrenergic receptors or ketamine at specific pH. The compositions preferably comprise brimonidine and/or dexmedetomidine and/or ketamine.

### CROSS-REFERENCE TO RELATED APPLICATIONS

This application is a continuation-in-part of U.S. patent application Ser. No. 12/798,925, filed Apr. 14, 2010, which is a continuation-in-part of U.S. patent application Ser. No. 12/460,970, filed Jul. 27, 2009, which claims a priority of U.S. Provisional Application Ser. Nos. 61/137,714, filed on Aug. 1, 2008; 61/192,777, filed on Sep. 22, 2008; 61/203,120, filed on Dec. 18, 2008; and 61/207,481 filed on Feb. 12, 2009. This application also claims a priority of U.S. Provisional Application Ser. No. 61/287,518, filed on Dec. 17, 2009. The contents of the above-mentioned applications are hereby incorporated by reference in their entirety.

### BACKGROUND OF THE INVENTION

Vascular Endothelial Growth Factor (VEGF) is an important molecule produced by cells which stimulates the growth of new blood vessels. Among other functions, VEGF maintains vascular integrity and is therefore a critical regulator responsible for maintaining the proper functioning of the blood vessels, including vascular permeability.

Vascular permeability may be divided into three basic categories: basal (i.e., normal physiologic functions); acute (associated with various pulmonary pathologies); and chronic (associated with such conditions as, for example, tumor angiogenesis, chronic hypoxia, and others).

In many pulmonary diseases and conditions (including but not limited to asthma, bronchiolitis, pneumonia, and others), acute vascular permeability is accompanied by an elevated level of VEGF. An elevated level of VEGF causes an inflammatory cascade, generally starting with vascular leakage and culminating with severe inflammation. Vascular leakage is primarily characterized by venular postcapillary leakage as a predominant component. This leakage involves several mechanisms and may include stripping of vascular endothelial cadherins (VE-cadherins) and formation of large endothelial gaps which leak plasma and proteins, including large proteins, such as fibrin. The leaking plasma, proteins and remaining exudative debris leak into bronchi and smaller bronchioles. There, they are exposed to clotting factors which precipitate large fibrin clots that further reduce ciliary mucous clearance and add mucous plugs. The cumulative result is inspissated (i.e., thickened/trapped) “secretions.” These accumulated secretions cause collapse of alveoli, further block mucous clearance, diminish alveolar gas exchange, attract water, solutes, and debris into the clots, and are very strong chemoattractants to neutrophils, promoting a strong inflammatory reaction as well as increasing the risk of infection due to stasis and reduced clearance of organic debris in the affected area(s).

The currently available anti-VEGF agents are ineffective and potentially deleterious for treating pulmonary diseases and conditions because they inhibit multiple and/or substantially all functions of VEGF, where such functions are multifactorial and considered essential for maintenance of normal vascular integrity within the lung. Thus, these anti-VEGF agents are ill-suited to treat pulmonary diseases and conditions.

Accordingly, there is a need for new compositions and methods that would reduce vascular permeability in pulmonary disease and inhibit harmful effects of elevated VEGF and its known potent induction of vascular permeability attendant to many such conditions without increasing the risk of untoward regulatory imbalance and/or deterioration of essential vascular integrity and homeostasis.

### SUMMARY OF THE PRESENT INVENTION

The present invention provides compositions and methods to treat and/or prevent pulmonary diseases and conditions (including, but not limited to asthma, pneumonia, edema, respiratory syncytial virus (RSV) disease, cystic fibrosis, acute respiratory distress syndrome, bronchiolitis, and acute lung injury) utilizing either: a) low concentrations of selective α-2 adrenergic receptor agonists which selectively constrict smaller blood vessels and/or b) ketamine at specific pH.

In some embodiments of the invention, the selective α-2 adrenergic receptor agonists have binding affinities (Kᵢ) for α-2 over α-1 receptors of 300:1 or greater. In preferred embodiments of the invention, the selective α-2 adrenergic receptor agonists have Kᵢ for α-2 over α-1 receptors of 500:1 or greater. In more preferred embodiments of the invention, the selective α-2 adrenergic receptor agonists have Kᵢ for α-2 over α-1 receptors of 700:1 or greater. In more preferred embodiments of the invention, the selective α-2 adrenergic receptor agonists have Kᵢ for α-2 over α-1 receptors of 1000:1 or greater. In even more preferred embodiments of the invention, the selective α-2 adrenergic receptor agonists have Kᵢ for α-2 over α-1 receptors of 1500:1 or greater.

In preferred embodiments of the invention, the selective α-2 adrenergic receptor agonists have binding affinities (Kᵢ) of 100 fold or greater for α-2b and/or α-2c receptors over α-2a receptors.

In preferred embodiments of the invention, the selective α-2 adrenergic receptor agonist is selected from the group consisting of brimonidine, dexmedetomidine, guanfacine, guanabenz, clonidine, and mixtures of these compounds.

In preferred embodiments of the invention, concentrations of the selective α-2 adrenergic receptor agonists are from about 0.001% to about 0.10%; more preferably, from about 0.001% to about 0.05%; even more preferably, from about 0.01% to about 0.025%; and even more preferably, from about 0.01% to about 0.02% weight by volume of the composition.

In preferred embodiments, the total dose of the selective α-2 agonist or ketamine delivered to a patient in need of treatment of a pulmonary disease or condition is between about 20 μg and 800 μg, more preferably between about 50 μg and about 500 μg, and still more preferably between about 200 μg and about 400 μg. The actual dose will depend on many factors, including but not limited to, the pH of the delivered drug, the lipophilicity, the particle size, and the delivery device employed. The total dose should be sufficient to create local absorption without causing systemic absorption.

In preferred embodiments of the invention, the selective α-2 agonist or ketamine have a relatively high lipophicity, with a Log P of between about 2.0 and about 4.5, and more preferably between about 2.5 and about 4.0.

Thus, in one embodiment, the invention provides a composition comprising a selective α-2 adrenergic receptor agonist having a binding affinity of 300 fold or greater for α-2 over α-1 adrenergic receptors, or a pharmaceutically acceptable salt thereof, wherein said α-2 adrenergic receptor agonist is present at a concentration from between about 0.001% to about 0.05% weight by volume.

In a preferred embodiment, a pH of the composition of the invention is between about 4.0 and about 8.5.

In one embodiment, the invention provides compositions comprising dexmedetomidine at pH of greater than about 7.1, more preferably between about 7.5 and about 8.5.

In another embodiment, the invention provides compositions comprising dexmedetomidine and ketamine at pH of between about 7.5 and about 8.5, and more preferably between about 8.0 and about 8.5.

In another embodiment, the invention provides compositions comprising a pharmaceutically effective amount of ketamine at pH of between about 7.5 and about 8.5, and more preferably between about 8.0 and about 8.5, for use in the treatment of pulmonary diseases or conditions, including but not limited to asthma, with reduced or eliminated systemic absorption of ketamine as compared to conventionally used ketamine formulations and/or doses. In this embodiment, the compositions do not need to contain selective α-2 agonists or other anti-asthma ingredients.

In a preferred embodiment, a pKa (a measure of the tendency to dissociate (ionize), which is related to pH for weak bases, such as α-2 agonists and ketamine: increased pH leads to reduced ionization and vice versa) of the composition of the invention is between about 5.0 and about 8.0.

If the primary symptoms of the condition being treated are associated with a serous discharge, luminal congestion, and/or mucosal leakage resulting in a severe cough (for example, viral upper respiratory tract infection (URI) and/or lower respiratory tract infection (LRI)), that is primarily related to the mucosal surface, then it may be desired to maintain pH of the composition between about 4.0 and about 6.5, more preferably between about 4.5 and about 6.0, and even more preferably between about 5.0 and about 5.5 and to shift equilibrium of the selective α-2 agonist to the ionized, less lipophilic state. Brimonidine or dexmedetomidine are the preferred selective α-2 agonists for the treatment of these conditions.

For some pulmonary diseases and conditions (for example, acute lung injury, respiratory distress syndrome, asthma, and others), it may be preferred to maintain pH of the composition between about 6.5 and about 8.5, more preferably between about 7.0 and about 8.0, and even more preferably between about 7.5 and about 8.5. Dexmedetomidine is the preferred selective α-2 agonist for the treatment of these conditions. As a result, the more alkaline end of the physiologically tolerated pH range creates an equilibrium of greater non-ionized form of the drug, with enhanced lipophilic absorption and mucosal membrane penetration into submucosa and muscularis. For highly lipophilic drugs, greater diffusion and depot absorption will result, particularly for drugs like dexmedetomidine that also have very high binding/retention to their membrane receptors (α-2).

In preferred embodiments, the compositions and methods of the invention may comprise magnesium sulfate and/or sodium citrate or its derivative (such as sodium citrate dehydrate or other pharmaceutically acceptable salt).

Preferably, the concentration of sodium citrate or its derivative is between about 0.05% and about 0.5%, more preferably, between about 0.01% and about 0.3%, and even more preferably, between about 0.15% and about 0.25%.

In one embodiment, the invention provides a composition comprising between about 0.01% to about 0.05% weight by volume of brimonidine, wherein pH of said composition is between about 4.0 and about 6.5.

In one embodiment, the invention provides a composition comprising between about 0.01% to about 0.10% weight by volume of dexmedetomidine, wherein pH of said composition is between about 7.0 and about 8.5. As there is a tendency for these drugs to create a more acidic pH than provided by their diluent, a buffer such as phosphate, borate or others well known to those skilled in the art is employed.

In some aspects, the compositions and methods of the invention further comprise other therapeutic agents, including bronchodilators and/or antibiotics.

In preferred embodiments, the bronchodilators may include, but are not limited to, selective and/or non-selective β-2 adrenergic receptor agonists, anticholinergics, and theophylline.

The invention also provides methods of treating and/or preventing a pulmonary disease or condition comprising administering to a patient in need thereof a therapeutically effective amount of the compositions of the invention.

In one embodiment, the invention provides methods of inducing a selective vasoconstriction of smaller blood vessels, such as microvessels, capillaries, and/or postcapillary venules relative to larger blood vessels, such as arteries and/or proximal arterioles. This selective vasoconstriction of smaller blood vessels allows for such effects while decreasing and/or eliminating ischemia risk. Unlike the present invention, α-1 agonists induce constriction of large and small vessels, for example causing constriction of the pulmonary artery. Therefore, α-1 agonists may considerably increase ischemia and secondarily inflammation. They are also direct agonist constrictors of bronchiole muscularis, which is equally or more damaging, since they cause direct bronchiole constriction, which is a highly deleterious and dangerous effect in respiratory compromised patients.

In accordance with the present invention, reduction of vascular permeability may reduce spread of viral and/or bacterial pathogens into surrounding lung parenchyma and may therefore reduce morbidity, and/or reduce the fibrin clotting, and mucus secretions resulting in inspissations and atelectasis, such as with pneumonia and/or a secondary pneumonia complicating an initial viral pneumonia. In this regard, the selective α-2 agonists being anti-sialogogues (reduce secretions), offer additional treatment benefits by allowing to avoid any significant α-1 receptor trigger.

In some aspects, the invention provides methods and compositions for treatment of pulmonary diseases and conditions that reduce or eliminate the need for steroids currently required in conventional treatments of pulmonary diseases and conditions. The steroid use can also decrease vascular permeability; however it usually requires many hours or even days for this decrease to be pronounced, with the maximum effect in many days or even weeks. This long time frame renders steroids not sufficiently active for the treatment of acute exacerbation of pulmonary conditions, whereas the α-2 agonist effect begins in minutes, peaks within hours and may have a duration of over ten hours for relatively lipophilic drugs such as dexmedetomidine.

In some aspects, the compositions of the invention also have anesthetic properties.

In some aspects, the compositions of the invention may be administered via aerosolized delivery and/or inhalation delivery.

### BRIEF DESCRIPTION OF THE FIGURES

FIG. 1 is a graphical representation of the factors causing airway obstruction in asthma patients;

FIG. 2 is a graphical representation of the effects of alveolar pneumonia;

FIG. 3 Is a graphical representation of the effects of brimonidine and dexmedetomidine on bronchoalveolar lavage (BAL) concentration of albumin;

FIG. 4 is a graphical representation of the effects of brimonidine and saline on central airway resistance in rats;

FIG. 5 is a chart of estimated sedation/local effects of nebulized dexmedetomidine at pH 5.0; and

FIG. 6 is a chart of estimated sedation/local effects of nebulized dexmedetomidine at pH 8.0.

### DETAILED DESCRIPTION OF THE INVENTION

### Definitions

The term “selective α-2 adrenergic receptor agonists” encompasses all α-2 adrenergic receptor agonists which have a binding affinity of 300 fold or greater for α-2 over α-1 adrenergic receptors. The term also encompasses pharmaceutically acceptable salts, esters, prodrugs, and other derivatives of selective α-2 adrenergic receptor agonists.

The term “low concentrations” refers to concentrations from between about 0.0001% to about 0.05%; more preferably, from about 0.001% to about 0.05%; even more preferably, from about 0.01% to about 0.025%; and even more preferably, from about 0.01% to about 0.02% weight by volume of the composition. In preferred embodiments, the above-mentioned concentrations are delivered over about 30 minutes. Because it is the total amount of the delivered compositions which is important, both concentrations and delivery time may be adjusted to arrive at roughly equivalent total amount of the delivered drug.

The term “brimonidine” encompasses, without limitation, brimonidine salts and other derivatives, and specifically includes, but is not limited to, brimonidine tartrate, 5-bromo-6-(2-imidazolin-2-ylamino)quinoxaline D-tartrate, Alphagan™, and UK14304.

The term “dexmedetomidine” encompasses, without limitation, dexmedetomidine salts and other derivatives.

The term “ketamine” encompasses, without limitation, ketamine salts, isomers, enantiomers and other ketamine derivatives, and specifically includes, but is not limited to, S-enantiomer of ketamine.

As used herein, the term “composition” is intended to encompass a product comprising the specified ingredients in the specified amounts, as well as any product which results, directly or indirectly, from a combination of the specified ingredients in the specified amounts.

The terms “treating” and “treatment” refer to reversing, alleviating, inhibiting, or slowing the progress of the disease, disorder, or condition to which such terms apply, or one or more symptoms of such disease, disorder, or condition.

The terms “preventing” and “prevention” refer to prophylactic use to reduce the likelihood of a disease, disorder, or condition to which such term applies, or one or more symptoms of such disease, disorder, or condition. It is not necessary to achieve a 100% likelihood of prevention; it is sufficient to achieve at least a partial effect of reducing the risk of acquiring such disease, disorder, or condition.

### EMBODIMENTS OF THE INVENTION

The invention provides compositions and methods to treat and/or prevent pulmonary diseases and conditions (including, but not limited to asthma, pneumonia, edema, respiratory syncytial virus (RSV) disease, cystic fibrosis, acute respiratory distress syndrome, bronchiolitis, and acute lung injury) utilizing either: a) low concentrations of selective α-2 adrenergic receptor agonists through selective inhibition of VEGF and/or b) ketamine at specific pH.

While not wishing to be bound to any particular theory, it is believed that VEGF plays a very important role in pulmonary diseases and conditions. VEGF levels are elevated in many, if not all, pulmonary diseases and conditions. Elevated VEGF levels cause postcapillary venular permeability increase and/or release of exudate which, among other consequences, may cause bronchiole obstruction, atelectasis (i.e., collapse of lung sacs), and other deleterious effects.

Further, it is believed that airway diseases, particularly asthma, have a multifactorial etiology. The current treatments are believed to act largely only at the level of anti-inflammatory and muscarinic active bronchial dilation and are not believed to adequately treat the cascade of effects resulting from triggers of antigens, allergens, pollutants, irritants, infection, concomitant nasal rhinitis, concomitant gastroesophageal reflux and others. In fact, in many cases asthma occurs without inflammatory pulmonary conditions, and vice versa, inflammatory pulmonary conditions may occur without asthma.

Further, as disease states such as asthma progress, airway remodeling results in edematous, thickened submucosa and muscularis layers, with thickening of the smooth muscle as well as increased density of angiogenic vasculature as well as distension and leakage of vessels, typically resulting in asthmatic condition refractory to steroids. Over 30% of asthmatics typically do not respond to steroids, and as this pathology is typically associated with such refractory states, these tend to be the more severe pathologies, and the more acute attacks, where hospitalization, and even mechanical ventilation, may be required as life saving measures, using β-2 agonists and inhaled corticosteroids available today. In such states conventional treatment is further compromised by hypersensitivy and excitability of the bronchial smooth muscle. Many β-2 agonists require the concurrent use of steroids. In more severe diseases, intravenous (IV) steroid use is necessary before conversion to inhaled steroids at a later time when the patient is more stable, and where time allows for the slow onset of inhaled steroid before systemic steroids are discontinued. Systemic steroids, and to a lesser extent inhaled steroids, have many systemic undesirable effects, including lowered infection resistance, glucose elevation, suppression of growth hormones and other hormones, etc.

Conventional α-1 agonists are poorly suited for treating pulmonary diseases and conditions because they cause constriction of both smaller and larger blood vessels, thereby contributing to ischemia. Exercise-induced asthma (EIA) which occurs in well over 50% of asthmatic patients, has been shown in numerous studies to have an associated morbidity with plasma norepinephrine levels, where norepinephrine has considerable α-1 activity. It is another advantage of selective α-2 agonists, particularly dexmedetomidine, to significantly suppress plasma and local norepinephrine levels at sub-sedation doses. Such suppression may reduce and/or alleviate ischemia.

A further advantage of selective α-2 agonists is their ability to suppress glutamate levels, which are known to cause excitoxicity to neurons, polymorphonuclear cells, and apoptosis, trigger NMDA receptors and further increase the morbidity associated with ischemia. The remodeled airway in chronic obstructive pulmonary disease and asthma may share many of the features of ischemic tissue, as hypoxia induces the remodeling due to poor vascular integrity, and where such edema causes muscle shortening and increased airway resistance. These effects may be alleviated by use of the present invention. The compositions and methods of the invention are able to counteract the deleterious effects of elevated VEGF levels without causing ischemia because they selectively cause constriction of smaller blood vessels (especially, postcapillary venules) while not affecting larger blood vessels.

It is a prophetic belief that polyanionic selective α-2 agonists effectively neutralize much of the pulmonary toxicity of eosinophilic infiltration, which is a major component of the morbidity of the pulmonary inflammatory reaction. Eosinophilic major basic protein cation has been found to contribute to pathology, including cytokine increase, vascular leakage, and bronchoconstriction.

In preferred embodiments, the compositions and methods of the invention result in reduced vascular permeability believed to be caused by postcapillary venular constriction induced by the inventive compositions and methods. Thus, the compositions and methods of the invention reduce the large VEGF-induced postcapillary venular gaps and related vascular permeability increase, resulting in selective inhibition of the acute vascular permeability increase and related inflammatory and hypoxic sequelae caused by elevated levels of VEGF. This postcapillary venular constriction is believed to be increased in hypoxic conditions typical of pulmonary pathology associated with VEGF increase.

It is believed that one of the reasons why the compositions and methods of the invention are effective in treating pulmonary diseases and conditions is because they are highly selective for α-2 adrenergic receptor agonists. Many α-2 agonists are also α-1 agonists, and therefore if α-2 agonists are insufficiently selective for α-2 receptors, they may have deleterious consequences associated with stimulating α-1 adrenergic receptors, causing profound vasoconstriction of large vessels contributing to ischemia, and/or inducing bronchiole constriction via α-1 muscularis receptors. Moreover, even selective α-2 adrenergic receptor agonists, when used at conventional doses of 0.1% or higher are associated with a number of undesirable side effects, such as rebound hyperemia and secondary vasodilation. These effects may be associated with a “cross-over” stimulation of α-1 adrenergic receptors as even the relatively low α-1 receptor stimulation versus α-2 receptor stimulation for these selective α-2 agonists becomes cumulatively significant at higher concentrations and α-1 agonist effects increasingly dominate because they are so untoward and potentially dangerous in these circumstances.

Additionally, it is believed that the selective α-2 agonists reduce multiple pathway triggers of pulmonary diseases, such as air pollutants, other irritants that trigger pain/afferent sensory neurons; and reduce the effects of stress and ischemia on pulmonary pathology; reduce airway reactivity and excitotoxicity.

Accordingly, in some embodiments, the invention utilizes selective α-2 agonists at low concentrations whereby α-2 receptor agonist activity is almost exclusively induced, and α-1 adrenergic receptors are not sufficiently stimulated to cause negative effects as described above.

In some embodiments, the preferred α-2 agonist is dexmedetomidine (log P is 2.98; the α2/α1 selectivity is over 1,500).

In some embodiments, the invention provides synergistic combinations of dexmedetomidine or other selective α-2 agonists with volatile anesthetics at subsedative levels, whereby these combinations provide synergistic effect for the treatment of asthma and/or other pulmonary diseases. For example, it is believed that the possible dexmedetomdine-induced bradycardia and hypotension may be offfset by ketamine. Because selective α-2 agonists and anesthetics have different mechanisms of action, there may be additional synergistic results from combining these two classes of drugs, with possible potentiation of effect and/or reduction of the required dose of either of the drug.

In some embodiments, the compositions and methods of the present invention can be used for the preventative treatment of asthma and other pulmonary disease. Specifically, the provided compositions and methods may suppress chronic triggers and their effects; reduce airway remodeling; and reduce mucosal edema.

It is another discovery of the invention that pH plays an important role in formulating the drugs for the treatment of pulmonary diseases. Specifically, if the drug's pKa is between 6.0 and 8.0 (i.e., the drug is a weak base), it should be preferably formulated at a more alkaline pH, so as to increase the amount of the drug in a non-ionized form, and therefore to improve its permeation hrough the respiratory mucosa and reduce the undesired systemic absorption of the drug and to lower its peak plasma concentration.

In one embodiment, the invention provides compositions comprising a pharmaceutically effective amount of ketamine at pH of between about 7.5 and about 8.5, and more preferably between about 8.0 and about 8.5, for use in the treatment of pulmonary diseases or conditions, including but not limited to asthma, with reduced or eliminated systemic absorption of ketamine as compared to conventionally used ketamine formulations and/or doses. In this embodiment, the compositions do not need to contain selective α-2 agonists or other anti-asthma ingredients.

It is believed that conventional formulations and/or dosages of ketamine were poorly suited for the treatment of asthma because they were accompanied by unacceptably high systemic absorption of ketamine which led to a host of negative side effects, such as sedation, dizziness, and others. The present invention overcomes these deficiencies by providing formulations of ketamine where its systemic absorption is minimized while its effectiveness in the treatment of asthma or other pulmonary diseases is maximized.

To treat asthma or other pulmonary diseases, ketamine may either be used together with selective α-2 agonists or by itself.

The pulmonary diseases and conditions that may be treated with the compositions and methods of the present invention include, but are not limited to, asthma, persistent asthma, status asthmaticus, as well as other forms of pulmonary diseases and conditions, including Methicillin-resistant Staphylococcus aureus (MRSA), strep, pneumoccal, viral and other forms of pneumonia, certain types of pulmonary edema, respiratory syncytial virus (RSV) disease, cystic fibrosis (particularly where bronchiectasis and/or atelectasis persist), acute respiratory distress syndrome, bronchiolitis, lung transplant rejection syndrome, acute lung injury, bronchial muscularis, viral upper respiratory tract infection (URI), viral lower respiratory tract infection (LRI), any condition associated with substantial mucosal discharge and/or submucosal/mucosal swelling and/or inflammation.

### Selective α-2 Adrenergic Receptor Agonists Suitable for the Purposes of the Invention

In some embodiments of the invention, the selective α-2 adrenergic receptor agonists have binding affinities (Kᵢ) for α-2 over α-1 receptors of 300:1 or greater. In preferred embodiments of the invention, the selective α-2 adrenergic receptor agonists have Kᵢ for α-2 over α-1 receptors of 500:1 or greater. In more preferred embodiments of the invention, the selective α-2 adrenergic receptor agonists have Kᵢ for α-2 over α-1 receptors of 700:1 or greater. In more preferred embodiments of the invention, the selective α-2 adrenergic receptor agonists have Kᵢ for α-2 over α-1 receptors of 1000:1 or greater. In even more preferred embodiments of the invention, the selective α-2 adrenergic receptor agonists have Kᵢ for α-2 over α-1 receptors of 1500:1 or greater.

The particularly preferred adrenergic receptor agonists for the purposes of the present invention have higher selectivity for α-2B and/or α-2C receptors, as compared to α-2A receptors within the lung. In preferred embodiments of the invention, the selective α-2 adrenergic receptor agonists have binding affinities (Kᵢ) of 100 fold or greater for α-2b and/or α-2c receptors over α-2a receptors. While not wishing to be bound to any specific theory, it is believed that α-2b receptors have the predominant peripheral vascular vasoconstrictive role in arterioles and venules. At the same time, α-2a receptors are predominantly found in the central nervous system, and therefore, α-2a specific agonists have a lesser role in causing direct vascular constriction and reduction of vascular permeability.

In cases of severe pulmonary compromise secondary to acute vascular permeability, including those that may be VEGF-induced, in addition to the α-2 and preferential α-2b agonist-induced microvessel terminal arteriolar and postcapillary venular constriction, a further advantage of the inventive compositions and methods may be activation of central nervous system (CNS) α-2a receptors. Activation of CNS α-2a receptors has been shown to have sedative effects, which may be beneficial in cases of bronchial constriction where anxiety and emotional stress are often contributing factors in cases refractory to treatment, and CNS α-2a receptors are also thought to be involved in a mechanism inducing bronchiole dilation.

The provided compositions may be modified to have higher concentration and/or smaller particle size, allowing both nonsedating and sedating/anxiolygic formulations, where the sedating/anxiolytic formulations take advantage of both high lung permeation and CNS manifested bronchodilation.

In preferred embodiments of the invention, concentrations of selective α-2 adrenergic receptor agonists are from about 0.001% to about 0.1%; more preferably, from about 0.001% to about 0.05%; even more preferably, from about 0.01% to about 0.025%; and even more preferably, from about 0.01% to about 0.02% weight by volume of the composition. In preferred embodiments, the above-mentioned concentrations are delivered over about 30 minutes. Because it is the total amount of the delivered compositions which is important, both concentrations and delivery time may be adjusted to arrive at roughly equivalent total amount of the delivered drug.

Any selective α-2 adrenergic receptor agonist with Kᵢ for α-2 over α-1 receptors of 300:1 or greater may be suitable for the purposes of the present invention. In preferred embodiments, Kᵢ for α-2 over α-1 receptors is 500:1 or greater, more preferably, 700:1 or greater, even more preferably 1000:1 or greater, and even more preferably 1500:1 or greater. In other preferred embodiments, the inventive compositions are more selective for α-2b receptors versus α-2a receptors.

Compositions and methods of the inventions encompass all isomeric forms of the described α-2 adrenergic receptor agonists, their racemic mixtures, enol forms, solvated and unsolvated forms, analogs, prodrugs, derivatives, including but not limited to esters and ethers, and pharmaceutically acceptable salts, including acid addition salts. Examples of suitable acids for salt formation are hydrochloric, sulfuric, phosphoric, acetic, citric, oxalic, malonic, salicylic, malic, furmaric, succinic, ascorbic, maleic, methanesulfonic, tartaric, and other mineral carboxylic acids well known to those in the art. The salts may be prepared by contacting the free base form with a sufficient amount of the desired acid to produce a salt in the conventional manner. The free base forms may be regenerated by treating the salt with a suitable dilute aqueous base solution such as dilute aqueous hydroxide potassium carbonate, ammonia, and sodium bicarbonate. The free base forms differ from their respective salt forms somewhat in certain physical properties, such as solubility in polar solvents, but the acid salts are equivalent to their respective free base forms for purposes of the invention. (See, for example S. M. Berge, et al., “Pharmaceutical Salts,” J. Pharm. Sci., 66: 1-19 (1977) which is incorporated herein by reference).

As long as a particular isomer, salt, analog, prodrug or other derivative of a selective α-2 adrenergic receptor agonist functions as a selective α-2 agonist, it may be used for the purposes of the present invention.

When choosing a particular α-2 adrenergic receptor agonist, one may take into account various considerations including any possible side effects and other systemic reactions.

In select circumstances, it may be preferable for the active agent of the present invention to penetrate parenchymal cell membranes, in which case a higher pH, including pH of greater than 7 may be desired. In this event, solubility may be reduced and require anionic components to stabilize. Such anionic components may include peroxide and/or other solubility enhancers and/or preservatives.

In preferred embodiments of the invention, the selective α-2 adrenergic receptor is brimonidine or its salt. In a more preferred embodiment, the selective α-2 adrenergic receptor agonist is the tartrate salt of brimonidine.

### Compositions and Methods of the Invention

In one embodiment, the invention provides a composition comprising a selective α-2 adrenergic receptor agonist having a binding affinity of 300 fold or greater for α-2 over α-1 adrenergic receptors, or a pharmaceutically acceptable salt thereof, wherein said α-2 adrenergic receptor agonist is present at a concentration from between about 0.001% to about 0.05% weight by volume.

In a preferred embodiment, the selective α-2 adrenergic receptor agonist is present at a concentration below about 0.05% weight by volume, and more preferably, between about 0.001% to about 0.05% weight by volume.

In one embodiment, the selective α-2 adrenergic receptor is selected from the group consisting of brimonidine, dexmedetomidine, guanfacine, guanabenz, clonidine, and mixtures of these compounds.

In a preferred embodiment, the composition comprises brimonidine at a concentration between about 0.001% and about 0.05% weight by volume.

In another preferred embodiment, the compositions of the present invention further include potassium (i.e., K⁺). The term “potassium” includes, but is not limited to, potassium salt. In another preferred embodiment, the compositions of the present invention further include calcium (i.e., Ca²⁺).

In a preferred embodiment, a pH of the composition of the invention is between about 4.0 and about 8.5.

In a preferred embodiment, a pKa (a measure of acidity) of the composition of the invention is between about 5.0 and about 8.0.

If the primary symptoms of the condition being treated are associated with a serous discharge, luminal congestion, and/or mucosal leakage resulting in a severe cough (for example, viral upper respiratory tract infection (URI) or lower respiratory tract infection (LRI)), then it is preferred that a pH of the composition is between about 4.0 and about 6.5, more preferably between about 4.5 and about 6.0, and even more preferably between about 5.0 and about 5.5.

For the treatment of these conditions, it is also preferred that a selective α-2 agonist is less lipophilic (lipophilicity may be measured, for example, by Log P or Log D values). Because less lipophilic selective α-2 agonists are preferred for the treatment of such conditions, brimonidine (Log P 0.45) is more preferred than dexmedetomidine (Log P 3.0).

There is a direct relationship between a selective α-2 agonist's lipophilicity (as characterized by the Log P or Log D values) and the pH of a pharmaceutical composition containing the selective α-2 agonist: as the pH increases across the range of 4.0 to 8.5, the selective α-2 agonist's lipophilicity exponentially increases. This correlation is true for virtually all selective α-2 agonists, and in particular, brimonidine and dexmedetomidine.

At a lower pH and a lower lipophilicity, bronchial mucosal membrane retention time is maximized and mucosal penetration is reduced. More of a drug is washed away and less is absorbed into lung parenchyma increasing the surface mucosal effect. The higher the lipophilicity, the greater is the selective α-2 agonist's penetration through the lipophilic mucosal cell membranes. This is because at a more alkaline pH, more of the compound is present in a non-ionized form. At pH of 6.7, about 65% of dexmedetomidine is ionized and about 35% is non-ionized; at pH of 8.0 or greater, about 10% of dexmedetomidine is ionized and about 90% is non-ionized, enhancing mucosal membrane penetration, diffusion, and, as prophetically believed, reducing systemic absorption into aqueous vessels.

When the pH is relatively low, e.g. between about 4.0 and about 6.5, the selective α-2 agonist is relatively less lipophilic and more ionized. As a result, a greater percentage of the selective α-2 agonist remains on the mucosa, increasing the drug's effectiveness as compared to the results at a higher pH.

For some pulmonary diseases and conditions (for example, acute lung injury, respiratory distress syndrome, asthma, and others), it may be preferred to maintain pH of the composition between about 6.5 and about 8.5, more preferably between about 7.0 and about 8.0, and even more preferably between about 7.5 and about 8.3.

For the treatment of these conditions, it is also preferred that a selective α-2 agonist is more lipophilic. Because more lipophilic selective α-2 agonists are preferred for the treatment of such conditions, dexmedetomidine (Log P 3.0) is more preferred than brimonidine (Log P 0.45).

At a higher pH and a higher lipophilicity, a greater penetration of the bronchial mucosa is achieved, allowing the selective α-2 agonist to reach submucosal vessels, glands, and/or nerves. A higher lipophilicity allows the drug to diffuse into lung parenchyma faster and more completely as compared to a lower lipophilicity. In addition, a greater mucosal penetration increases local efficacy and reduces systemic absorption (this is believed to be primarily due to the decreased oropharyngeal and gastrointestinal tract absorption). More of a drug is a non-ionized form, allowing for a quick and more complete diffusion through lung tissue and lipophilic membrane tissue, resulting in a high local retention and a very low systemic absorption.

When a selective α-2 agonist is lipophilic, it becomes highly ionized as it enters the distal esophagus and stomach. Once there, any lipophilic drug with a pKa of 5.5 or greater is essentially 100% ionized. Accordingly, this drug will have reduced lipophilic membrane penetration and low systemic absorption. For example, about 65% of dexmedetomidine is ionized in pharynx (pH is about 6.8), and virtually 100% ionized in stomach (pH of less than 5.0). Thus, it is very poorly absorbed in lipophilic membranes of the gastrointestinal tract and therefore, results in less systemic absorption.

In contrast, pH environment in the lungs (and in airways) is about 7.4 to 7.8, whereby only 10-23% of dexmedetomidine is ionized, therefore allowing the drug to better penetrate respiratory mucosa.

In one embodiment, the invention provides a composition comprising between about 0.003% to about 0.025% weight by volume of dexmedetomidine, between about 0.05 to about 5 mM of magnesium chloride, between about 1 mM to about 50 mM of potassium chloride and wherein pH of said composition is between about 4.0 and about 8.5. In a more preferred embodiment, the composition has pH of between about 7.8 and about 8.2.

In some aspects, the compositions and methods of the invention further comprise other therapeutic agents, including bronchodilators and/or antibiotics.

In preferred embodiments, the bronchodilators may include, but are not limited to, β-2 adrenergic receptor agonists, anticholinergics, and theophylline.

In one embodiment, the compositions and methods of the invention comprise ketamine, which is a potent intravenous sedative. While it has been known that ketamine is an antagonist of NMDA receptor, and can be used as a bronchodilator, it has been associated with an undesirable and unpleasant form of sedation, known as dissociative sedation. Dissociative sedation may be associated with a conscious vacant stare effect, reduction in short term memory, and multiple other neurologic consequences of sedation which are undesired in the treatment of a patient with a pulmonary disease.

Typically, sedation occurs at ketamine plasma levels of about 9000 μg/L. However, dissociative changes have been documented at ketamine plasma levels of 1000 ug/L or below. Prior art attempts to use inhalation forms of ketamine to increase the local effect and reduce systemic effect have resulted in over 800 ug/L plasma levels which is too high.

It is a discovery of the present invention that at pH of about 8.5, the ionization of ketamine decreases to about 10%, from over 60% at physiologic pH. Without wishing to be bound to a particular theory, it is believed that at pH of higher than about 7.6, the resultant reduction in ionization of ketamine allows for improved bronchial wall and lung tissue permeation with reduced vascular absorption. Accordingly, it is possible to achieve maximum local effects sufficient to treat pulmonary diseases with ketamine plasma levels of well below 800 ug/L, and therefore, with minimized or absent undesirable clinically appreciable dissociative effects.

Accordingly, in one embodiment, the invention provides a composition comprising a selective α-2 adrenergic receptor agonist having a binding affinity of 1000 fold or greater for α-2 over α-1 adrenergic receptors, or a pharmaceutically acceptable salt thereof, wherein said α-2 adrenergic receptor agonist is present at a concentration from between about 0.001% to about 0.05% weight by volume, and further comprising a pharmaceutically effective amount of ketamine, wherein pH of said composition is between about 7.5 and about 8.5; more preferably, between about 7.8 and about 8.2.

It is also believed that dexmedetomidine and ketamine have synergistic analgesic effects. It is further believed that the compositions including selective α-2 agonists and ketamine may have synergistic effects in reducing inflammation, reducing C fiber sensory neuron neuropeptide release, suppression of reactive oxygen species creation, and reducing polymorphonuclear neutrophils (PMN) recruitment.

In another embodiment, the invention provides a composition comprising a pharmaceutically effective amount of ketamine, wherein pH of said composition is between about 7.5 and about 8.5; more preferably, between about 7.8 and about 8.2.

In one embodiment, the invention provides a composition comprising between about 0.01% to about 0.05% weight by volume of brimonidine, wherein pH of said composition is between about 4.0 and about 6.5.

The invention further provides a method of treatment of a pulmonary disease in a patient in need thereof comprising administering to said patient a composition comprising between about 0.01% to about 0.05% weight by volume of brimonidine, wherein pH of said composition is between about 4.0 and about 6.5, wherein the composition is administered for about 5 to about 30 minutes.

In one embodiment, the invention provides a composition comprising between about 0.01% to about 0.025% weight by volume of dexmedetomidine, wherein pH of said composition is between about 4.0 and about 6.5.

The invention further provides a method of treatment of a pulmonary disease in a patient in need thereof comprising administering to said patient a composition comprising between about 0.01% to about 0.025% weight by volume of dexmedetomidine, wherein pH of said composition is between about 4.0 and about 6.5, wherein the composition is administered for about 5 to about 30 minutes.

If the concentrations of the provided compositions are changed, then the total administration time is changed accordingly to provide a roughly equivalent amount of the total delivered composition.

The compositions of the present invention are preferably formulated for a mammal, and more preferably, for a human.

The invention also provides methods of treating and/or preventing a pulmonary disease or condition comprising administering to a patient in need thereof a therapeutically effective amount of the compositions of the invention.

In preferred embodiments, the compositions and methods of the invention cause postcapillary venular constriction thus counteracting the clinically damaging increase in acute vascular permeability caused by elevated levels of VEGF. While VEGF is a primary pro-inflammatory cytokine therapeutically altered by the present invention, other cytokines, such as interleukin 6, 13, and serotonin, to name a few, may be similarly affected.

In some embodiments, the invention provides methods of inducing a selective vasoconstriction of smaller blood vessels, such as microvessels, capillaries, and/or postcapillary venules relative to larger blood vessels, such as arteries and/or arterioles. This selective vasoconstriction of smaller blood vessels allows decreasing and/or eliminating ischemia.

In some aspects, the invention provides methods and compositions for treatment of pulmonary diseases and conditions that reduce or eliminate the need for steroids currently required in conventional treatments of pulmonary diseases and conditions.

In some aspects, the compositions of the invention may be administered via aerosolized delivery and/or inhalation delivery.

In some aspects, the compositions of the invention also have anesthetic properties.

### Aerosolized and Nebulized Compositions

In preferred embodiments, the compositions of the invention are aerosolized or nebulized. In one embodiment, the aerosolized or nebulized composition is formulated for treating and/or preventing a pulmonary condition. It is within a skill in the art to prepare the aerosolized compositions of the present invention.

The aerosolized or nebulized compositions of the present invention are generally delivered via an inhaler, jet nebulizer, or ultrasonic nebulizer which is able to produce aerosol particles with size of between about 1 and 10 μm.

In one embodiment, the invention provides an aerosolized composition comprising a selective α-2 adrenergic receptor agonist having a binding affinity of 300 fold or greater for α-2 over α-1 adrenergic receptors, or a pharmaceutically acceptable salt thereof, wherein said α-2 adrenergic receptor agonist is present at a concentration from between about 0.001% to about 0.05% weight by volume.

In one embodiment, the selective α-2 agonist may be formulated in about 5 ml solution of a quarter normal saline having pH between 4.5 and 6.5, preferably between 4.5 and 6.0, to be delivered over about 5 to 30 minutes or such time so as to provide an equivalent total amount of the delivered drug if the concentrations are changed.

In a preferred embodiment, the aerosolized or nebulized composition comprises about 0.02% weight by volume of brimonidine in about 5 ml solution, which further comprises about 0.225% weight by volume of sodium chloride, and wherein said composition has a pH between about 4.5 and about 6.5, to be delivered over 5 to 30 minutes or such time so as to provide an equivalent total amount of the delivered drug if the concentrations are changed.

In some embodiments, particularly for the treatment of influenza and other pulmonary pathogen infections, the aerosolized or nebulized compositions are delivered in sufficient concentrations to create effective systemic/local tissue as well as local mucosal concentration of the drug.

In preferred embodiments, the aerosolized or nebulized compositions are delivered at sufficient concentration and in sufficient duration to create effective systemic/local tissue as well as local mucosal concentration of the drug.

In some embodiments, the aerosolized or nebulized compositions are effective for systemic effect on the central nervous system.

In some embodiments, the aerosolized or nebulized compositions are effective for treating pulmonary disorders or conditions.

In some embodiments, the invention provides a method of treating influenza and/or a secondary lung infection comprising administering to a patient in need thereof a selective α-2 adrenergic receptor agonist, or a pharmaceutically acceptable salt thereof, wherein said selective α-2 adrenergic receptor agonist is present at a concentration below about 0.05% weight by volume.

In some embodiments, the invention provides a method of treating ambulatory asthma or upper/lower respiratory congestion by administering to a patient in need thereof a metered dose of a composition comprising 0.05% by weight of brimonidine or dexmedetomidine via an inhalant.

In some embodiments, the invention provides a method of treating influenza, status asthmaticus, or persistent severe asthma by administering to a patient in need thereof a nebulized composition comprising 0.05% by weight of brimonidine or dexmedetomidine for about 5 to 30 minutes, or such time so as to provide an equivalent total amount of the delivered drug if the concentrations are changed.

In some embodiments, the total amount of ketamine delivered to a patient is between about 5 μg and about 500 μg, more preferably, between about 10 μg and about 150 μg; more preferably, between about 50 μg and about 100 μg.

In other embodiments, the total amount of ketamine delivered to a patient is between about 20 μg and about 1000 μg, more preferably, between about 100 μg and about 800 μg; more preferably, between about 200 μg and about 600 μg.

In some embodiments, the invention provides a method of treating a pulmonary disease or condition comprising administering to a patient in need thereof a pharmaceutically effective amount of ketamine, wherein pH of said composition is between about 7.0 and about 8.5, wherein said composition does not contain other active agents effective for the treatment of said pulmonary disease or condition, and wherein said ketamine is nebulized at between 12.5 to about 25 mg/ml, and wherein said composition is delivered for about 20 to about 30 minutes.

In preferred embodiments, ketamine is nebulized at about 0.01% and is delivered for about 5 to about 30 minutes, or such time so as to provide an equivalent total amount of the drug delivered if the concentrations are changed.

In other preferred embodiments, ketamine is nebulized at about 0.03% and is delivered for about 3 to 15 minutes, or such time so as to provide an equivalent total amount of the delivered drug if the concentrations are changed.

In some embodiments, the secondary drug infection is a pneumococcal infection.

In a preferred embodiment, the invention provides a method of treating influenza and/or pneumococcal infection comprising administering to a patient in need thereof about 0.05% weight by volume of a selective α-2 adrenergic receptor agonist, wherein said selective α-2 adrenergic receptor agonist is nebulized. Preferably, the treatment time is about 5 to 30 min, or such time so as to provide an equivalent total amount of the delivered drug if the concentrations are changed.

In general, conditions that are less acute may be treated via metered dose by inhaler, including cases of asthma, upper and lower respiratory congestion and walking pneumonia. Conditions that are more acute may require nebulized drug. Such conditions include but are not limited to persistent asthma, status asthmaticus, viral and/or bacterial pneumonia, respiratory syncytial virus disease, infant bronchiolitis, and acute lung injury.

Compositions for Oral and/or Intravenous Administration

In some embodiments, the compositions of the present invention can be included in a pharmaceutically suitable vehicle suitable for oral ingestion. Suitable pharmaceutically acceptable carriers include solid fillers or diluents and sterile aqueous or organic solutions. The active compound is present in such pharmaceutical compositions in an amount sufficient to provide the desired effect.

The solubility of α-2 agonists decreases exponentially at an increased pH. Table 1 illustrates the relationship between pH and solubility in water for dexmedetomidine. It shows that the soluble concentration of dexmedetomidine falls exponentially with higher pH.

TABLE 1

| pH | solubility (mg/ml) | max soluble concentration | BSS solution* |
|---|---|---|---|
| 6.0 | 1.953 | 0.195% | |
| 6.4 | ~0.60 | 0.060% | |
| 7.0 | 0.224 | 0.023% | ≧0.10% |
| 7.4 | ~0.150 | 0.015% | |
| 8.0 | 0.134 | 0.013% | |

*BSS = Balanced Salt Solution

In some embodiments of the present invention, it may be necessary to improve (i.e., increase) the solubility of α-2 agonists. A greater solubility has a number of advantages, including but not limited to an ability to achieve higher concentrations and enhanced stability at storage at cold temperatures. Because the desired concentration of suitable α-2 agonists is very low, and the present invention provides formulations with much greater solubility, the desired concentrations are easily achieved even at an exponentially reduced known solubility in the desired near-alkaline to alkaline pH range.

It is a surprising discovery of the present invention that α-2 agonists, and more specifically, dexmedetomidine, are rendered more effective as well as more soluble by constituents of a balanced salt solution. The terms “salt” and “constituent of a balanced salt solution” are used interchangeably for the purposes of the present invention. They are a subset of agents that improve solubility of the inventive formulations.

Thus, in one embodiment of the present invention, dexmedetomidine is rendered soluble up to or beyond 0.1% at pH 7.1 by adding constituents of a balanced salt solution. In a preferred embodiment, these constituents include any combination of one or more of the following: sodium citrate dehydrate, sodium acetate, and calcium salt. In a more preferred embodiment, the concentration of sodium dehydrate is about 0.17%; the concentration of sodium acetate is about 0.39%; and the concentration of calcium salt is about 0.048%.

The most preferred agent that improves solubility is a citrate salt. Citrate salt acts as a preservative and a corneal penetration enhancer. It has been further discovered that to achieve the desired final pH of around 6.4 to 6.7 and Log D values of above 2.50 of dexmedetomidine formulations, one may add a citrate salt at pH of about 7.1, preferably using an acetate or other similar pH range buffer.

Other agents that improve solubility which may be used for the purposes of the present invention include, but are not limited to, methanesulfonate (mesylate), hydrobromide/bromide, acetate, fumarate, sulfate/bisulfate, succinate, citrate, phosphate, maleate, nitrate, tartrate, benzoate, carbonate, pamoate, borate, glycolate, pivylate, sodium citrate monohydrate, sodium citrate trihydrate, sodium carbonate, sodium EDTA, phosphoric acid, penatsodium pentetate, tetrasodium etidronate, tetrasodium pyrophosphate, diammonium ethylenediamine triacetate, hydroxyethyl-ethylenediamine triacetic acid, diethylenetriamine pentaacetic acid, nitriloacetic acid, and various other alkaline buffering salts, polyanionic (multiple negatively charged) compounds, such as methylcellulose and derivatives, particularly carboxymethyl cellulose (CMC); and/or addition of cyclodextrins and/or their derivatives, particularly (2-Hydroxypropyl)-beta-cyclodextrin; certain solvents such as Tween 20, Tween 80, polyvinyl alcohol, propylene glycol and analogues or derivatives thereof; certain osmotic agents, such as mannitol or sucrose, HPMC or analogues and/or derivatives thereof, or certain chelating agents.

It is well within a skill of a skilled in the art to determine the amounts and concentrations of the agents improving solubility.

Pharmaceutical compositions contemplated for use in the practice of the present invention can be used in the form of a solid, a solution, an emulsion, a dispersion, a micelle, a liposome, and the like, wherein the resulting composition contains one or more of the active ingredients in admixture with an organic or inorganic carrier or excipient suitable for nasal, enteral, or parenteral applications.

The active ingredients may be combined, for example, with the usual non-toxic, pharmaceutically and physiologically acceptable carriers for tablets, pellets, capsules, troches, lozenges, aqueous or oily suspensions, dispersible powders or granules, suppositories, solutions, emulsions, suspensions, hard or soft capsules, caplets or syrups or elixirs and any other form suitable for use. The possible carriers include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents may be used.

In yet another embodiment, the compositions of the present invention may be formulated for an intravenous (IV) administration. It is within a skill in the art to formulate the compositions for an IV administration.

Diseases and Conditions to be Treated with Compositions and Methods of the Invention

The invention provides compositions and methods that may be used to treat or prevent a variety of pulmonary diseases and conditions. Pulmonary diseases and conditions include, but are not limited to cystic fibrosis and various other forms of pulmonary diseases and conditions, including edemas (including interstitial edema, pulmonary edema, mucosal edema, submucosal edema and other edemas), vascular congestion, mucosal swelling of bronchi and bronchioles, infectious tracheobronchitis, respiratory syncytial virus (RSV) bronchitis, etc. Other pulmonary uses include treatments of increased vascular leakage/permeability that further swell the bronchiole mucosa and shrink the available lumen size of an airway. Such increases in vascular permeability occur in a disease of the respiratory tract, allergic rhinitis, common cold; influenza; asthma, exercise induced asthma, asthma-associated rhinitis, asthma-associated gastro-esophageal reflux; any disease associated with bronchiospasms, acute respiratory distress syndrome, and acute lung injury.

Such conditions produce added increase in generalized inflammation and secretions with high morbidity in such conditions as pneumonia, and cystic fibrosis after secondary pneumonia, as often occurs with secondary pneumococcal and other infections. Such conditions can cause alveolar capillary increased permeability and capillary changes along the mucosal surface that swell the mucosa into the lumen. An increase in vascular permeability is known as one of the main features by which these pathogens are disseminated inside a host organism through cascade of inflammatory byproducts and other specific means of induction. In addition, the compositions and methods of the inventions can be used to treat patients after undergoing sinus surgery.

Unlike prior art pulmonary treatment for lung pathologies, in which a specific drug is applicable to a single or limited pathologies, the present invention allows for unique properties specific to α-2 agonists to be effective in the entire spectrum of respiratory conditions, from those affecting the proximal oropharynx, to the most distal lung parenchyma.

Three specific attributes are optimized by the combination of high selectivity and low concentration that results in α-2 receptor trigger without untoward α-1 receptor activation: 1) the anesthetic properties of α-2 agonists; 2) the vasoconstrictive-vascular permeability reducing properties; and 3) the CNS properties of sedation and bronchiole dilation.

By offering a spectrum of particle sizing, inhalation vehicles, and flow rates an expert in the art can provide treatment with the present invention localized to primarily the distribution best for that specific entity, ranging from oropharynx for pharyngeal inflammation (sore throat), upper respiratory tract conditions (URI); lower respiratory tract conditions (LRI); known distal distribution of bronchiole B2 receptors to achieve primarily luminal relief for congested/constricted airways; to the most distal, lung parenchyma, as with viral and infectious conditions that have primarily or secondarily reached beyond and/or far beyond the respiratory tract itself to systemic absorption.

Particle sizing is typically in the range of 1 μm to 8 μm, where the smallest size droplets reach most distal into lung parenchyma and the largest most proximal. For the present invention this is extended to about 0.01 μm to about 20 μm, allowing for a more complete range of proximal to distal droplet anatomic targeting as the present invention can utilize. A dispenser designed to allow an adjusted or multiple sized combination with a range of 0.01 μm to 50 μm, and more preferably 0.05 μm to 20 μm, and still more preferably 1 um to 10 um could be used to allow for treatment of a variety of conditions, for example proximal spectrum largest droplet size: pharyngitis+upper respiratory airway disease; midspectrum mid range droplet size: lower respiratory tract+B2 selective distribution; to distal spectrum smallest droplet size: lung parenchyma and maximal systemic absorption for CNS distribution at the other end of the spectrum.

For treatment of specific conditions with the present invention where bronchiole constriction is the primary pathophysiologic process, such as for asthma treatment, use of highly selective alpha 2 agonists, with binding affinity of 300 more higher, along with concentration of 0.001-0.05% and particle size of preferably 2-7 μm, more preferably 2.5-5.0 μm, and more preferably 2.0-3.0 μm is recommended. Aerosol should be delivered to the alveoli if delivery to the circulatory system is desired, using particle sizes in the smaller droplet of this range, provided the particle has a density similar to water, and a generally spherical shape. Particles with higher or lower density will effectively behave as bigger or smaller particles, respectively. Diseases of small airways and alveoli (e.g., asthma, emphysema, pulmonary infections, etc.) may similarly require delivery with small, typically 1-2 μm, spherical particles. Therefore, the present invention allows for and can be optimized for targeted delivery to areas of bronchiole constriction to reduce luminal congestion, as well as CNS absorption to reduce anxiety and through CNS trigger reduce bronchiolar constriction as well. A particle size of 2.5 μm combined with one of 1.5 μm or less allows for both bronchiolar areas and CNS absorption.

For treatment of pneumonia, bacterial or viral where lung parenchyma absorption is the primary pathophysiologic process, use of highly selective alpha 2 agonists, with binding affinity of 300 or higher, along with concentration of 0.001-0.059% and particle size of preferably 0.5-3 μm, more preferably 1-2.5 μm, and still more preferably 1.5 μm-2.0 μm is recommended. Utilizing higher pH, such as between 6.5 and 8.0, and more preferably 6.8 to 7.5, and still more preferably 7.0-7.25 allows for greater lipophilic cell membrane permeabilities, as occurs in cell walls of lung parenchymal tissue to still further increase such desired absorption. As solubility of alpha 2 agonists decreases with pH at or above 7.0 some solubility enhancement as known to experts in the art, such as with anionic stabilizers and/or preservatives such as peroxide based compounds may be added, however the low concentrations desired for the highly selective alpha 2 agonists per this invention minimizes this need to one of possible preference and mild enhancement of efficacy rather than necessity, one also dependent on the concentration within the range of the invention desired.

It has been further documented that in patients following sinus surgery increased access to and absorption within the sinus cavities results, where particle sizes from 1 μm to 25 μm may be effectively absorbed. This is particularly true for the maxillary sinus after maxillary sinuses after maxillary antrostomy and ethmoidectomy. Treatment with the present invention in the postoperative state can reduce swelling, inflammation, and related morbidity.

Particle size can be controlled by a variety of means, such as use of porous membranes of various pore dimensions. Further applying energy to reduce the bulk of aerosolized media may optionally be employed to enhance the percentage of smaller particle sizes as desired; pore size of the aerosolization membrane; temperature of aerosolization; extrusion velocity; ambient humidity; the concentration, surface tension, viscosity of the formulation; and vibration frequency.

Aerosol particle size can be adjusted by adjusting the size of the pores of the membrane, as discussed, for example, in U.S. Pat. No. 7,244,714.

Drug delivery is dependent on several variables well known to experts in the art. The delivery device depends on factors including but not limited to a particular preferred drug, the particle size, the drug's Log P value, pKa, preferred pH range, concentration, binding receptor strength to its intended receptor, tissue diffusion profile, systemic effects, racemic vs. enantiomer preference, etc. Therefore, specific devices and specific ranges for drug delivery will depend on these key parameters.

To optimize local lung distribution while minimizing systemic distribution, a particle size of between 2 and 5 microns is preferred. Typically only 8-15% of the drug contained in the nebulizer ultrasonic bath is delivered to a patient.

The more lipophilic the drug is, and quicker its tissue diffusion, the less likely it is to trigger α-1 receptors, and therefore, higher concentrations can be used. It is the total dose of the delivered drug, rather than concentration and the drug's deposit density that are more important to avoid triggering α-1 receptors.

Typically, the preferred total dose of a selective α-2 agonist and/or an anesthetic, such as ketamine, is between at least 10 μg and not above 1000 μg (1 mg). In a preferred embodiment, the dose is between about 50 μg and about 500 μg, and in an even more preferred embodiment, the dose is between about 200 μg and about 400 μg.

Further, when a selective α-2 agonist and ketamine are used in combination, lower doses of both drugs will be required due to their expected synergistic effects. For a nebulized dose of for example 0.05%, it is anticipated that between about 5 min and about 20 min of nebulized delivery administration time will be sufficient. The concentration may be varied: for example, if the concentration is doubled, then the administration time may be halved, and vice versa.

The present invention is more fully demonstrated by reference to the accompanying drawings.

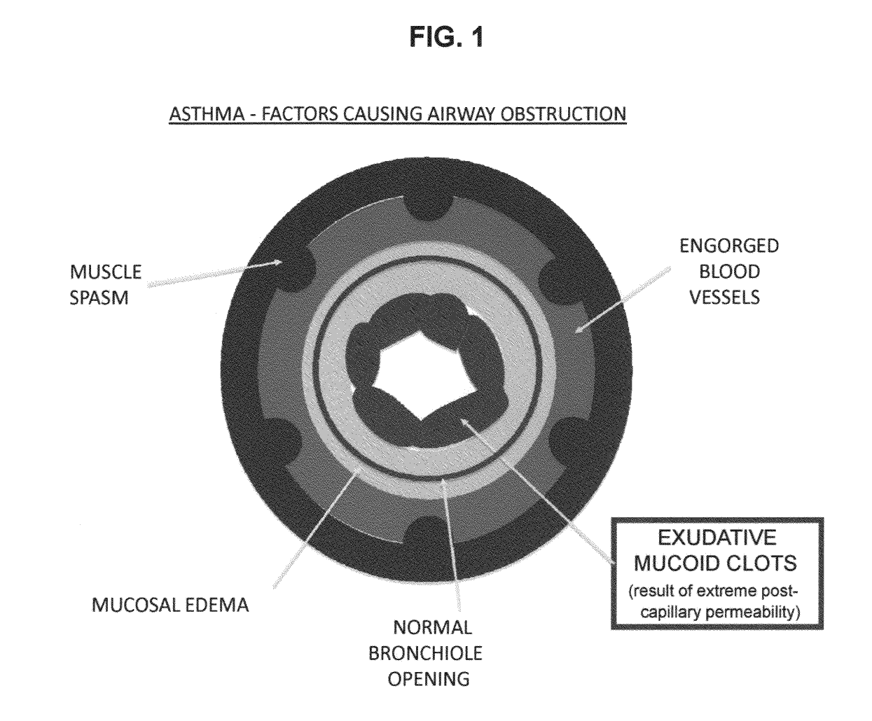

FIG. 1 is a graphical representation of the factors causing airway obstruction in asthma patients. As FIG. 1 demonstrates, these factors include muscle spasm, mucosal edema, engorged blood vessels, and exudative mucoid clots. These clots are a result of extreme postcapillary venular permeability believed to be increased at the site of action, and causally related to (or, at the very least contributed to) elevated VEGF levels.

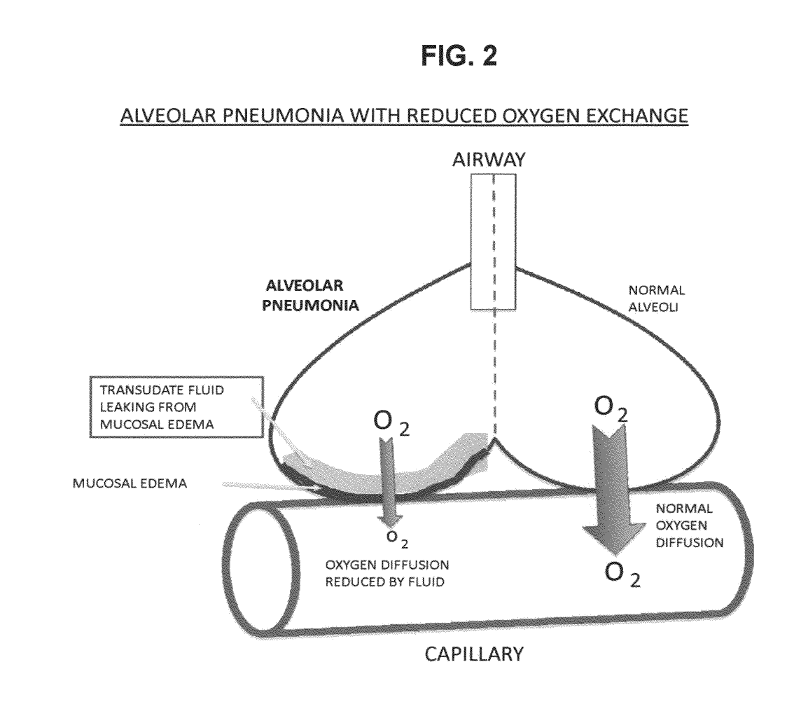

FIG. 2 is a graphical representation of the effects of alveolar pneumonia. As FIG. 2 demonstrates, in alveolar pneumonia, transudate fluid is leaking from mucosal edema, causing reduction in oxygen diffusion in the capillaries.

The following Examples are provided solely for illustrative purposes and is not meant to limit the invention in any way.

### Example 1

### Effect of Brimonidine and Dexmedetomidine on the Levels of Albumin in Airspace Following Exposure to an Allergen

The purpose of this experiment was to determine if there is any effect from pre-treating animals exposed to allergens with brimonidine and dexmedetomidine. IgE-mediated immediate hypersensitivity reactions involve mast cell degranulation; and the released mediators cause plasma extravasation from the post-capillary venules. Therefore, exposure to a pulmonary allergen should increase the concentration of albumin in the airspace within a few minutes of allergen exposure.

### Experimental Design

16 adult Brown Norway (BN) rats were sensitized to ragweed pollen extract, and randomized to 4 groups: control, brimonidine, dexmedetomidine, and saline.

The control group rats were pretreated with aerosolized saline and administered aerosolized saline. The saline group rats were pretreated with aerosolized saline, then exposed to aerosolized ragweed extract for 10 minutes. The brimonidine group rats were exposed to aerosolized 0.05% brimonidine for 10 minutes, followed by exposure to aerosolized ragweed extract for 10 minutes. The dexmedetomidine group rats were exposed to aerosolized 0.005% dexmedetomidine for 10 minutes, followed by exposure to aerosolized ragweed extract for 10 minutes.

At 10 minutes after completion of the allergen exposure, rats were anesthetized and exsanguinated, and their lungs were lavaged with 50 ml of buffer solution. The resulting bronchoalveolar lavage (BAL) fluid was centrifuged to remove the cells, and the supernatants were analyzed for albumin concentration using an ELISA assay for rat albumin.

### Results

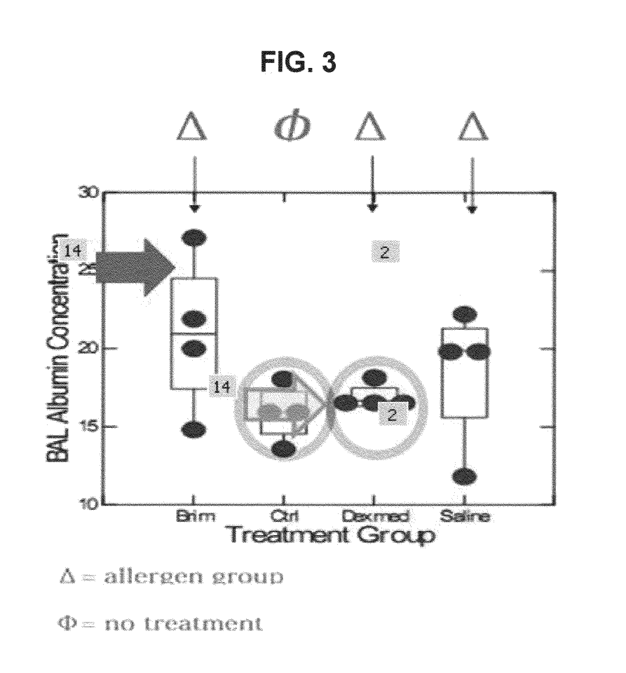

FIG. 3 demonstrates BAL albumin concentrations for each treatment group. While there are no statistically significant differences among groups, due to the variability of allergic responses and the small numbers of rats, most of the rats that were pretreated with saline or brimonidine had elevated BAL albumin compared with control rats. It appears therefore that brimonidine had no apparent protective effect. On the other hand, dexmedetomidine group rats had albumin levels similar to those of control rats, suggesting a protective effect of dexmedetomidine.

### Example 2

### Effect of Brimonidine vs Saline on Airway Secretions in Inflamed Lungs

The purpose of this experiment was to compare the effect of brimonidine vs saline on the amount of airway secretions in inflamed lungs of rats. The experiment was designed as follows. 10 rats were administered either saline solution (6 rats) or brimonidine at 200 μg/ml (0.02%), 400 μg/ml (0.04%), and 800 μg/ml (0.08%) (4 rats).

The resistance at the first time point prior to administration of saline or brimonidine was established at 100%, establishing the baseline. The mean resistance at baseline was similar for the two treatment groups, and therefore, all the measured resistances were expressed as a % of the baseline resistance. After establishing baseline conditions, the first aerosol treatment (saline or brimonidine at 200 μg/ml) was delivered for one minute, followed by 10 minutes of monitoring. The airway resistance at the end of the 10-minute period was the first post-treatment resistance, and the airway resistance measured immediately after removal of tracheal secretions was the second post-treatment resistance. These measurements were repeated for two more aerosol treatments in each rat.

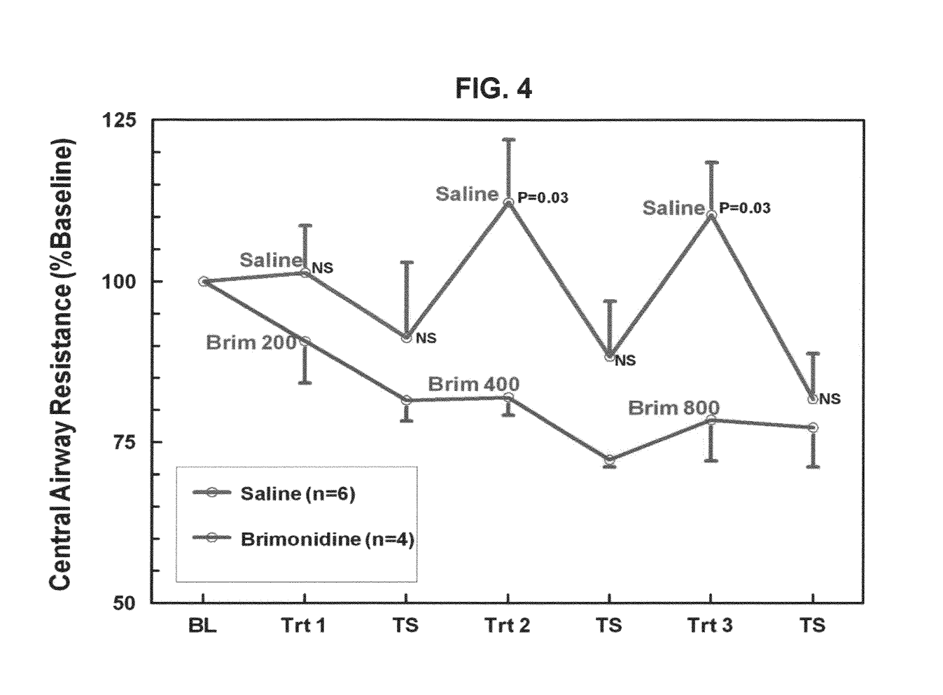

The 2 groups were compared statistically at various post-treatment time points. FIG. 4 is a plot of Central Airway Resistance (% of baseline) vs various time points. Trt 1, Trt 2, and Trt 3 denote 10-minute periods immediately after first, second, and third treatments, respectively. TS denotes a time point after tracheal secretions were removed after the treatments.

Results

In the saline-treated rats, there is an increase in resistance after 10 minutes after each aerosol treatment (i.e. at Trt 1, Trt 2, and Trt 3). However, the effect is completely reversed after tracheal suctioning (i.e., at TS points). This suggests that the increases in resistance are due to accumulating secretions. However, in the brimonidine-treated rats, there is little increase in resistance during the post-treatment monitoring period, and little change after suctioning. Statistically, the post-treatment resistances are significantly higher (P=0.03) in the saline group compared with the brimonidine group after the 400 and 800 μg/ml doses, but there is not a significant difference between groups for the post-suctioning resistances at any of the time points. Further, the final pre/post-suctioning change in resistance is significantly greater in the saline group than the brimonidine group (P=0.006).

These observations are consistent with brimonidine reducing the accumulation of airway secretions in inflamed lungs. The lack of a significant difference in post-suctioning resistances between the saline and brimonidine groups suggests that brimonidine did not alter airway mucosal edema substantially in the central airways.

### Example 3 (Prophetic)

### Effect of Brimonidine and Dexmedetomidine on Inhibition of VEGF Inflammatory Cascade

The purpose of this experiment is to test the effect of administering aerosolized brimonidine and dexmedetomidine on pulmonary function in acute respiratory viral infection.

Study Design

A parallel group design of five groups of eight rats each: virus/saline, virus/brimonidine, virus/dexmedetomidine, sham/saline, sham/brimonidine. Treatments are twice daily, beginning one day post inoculation, and ending the morning of terminal studies on day 4, 5 or 6 post inoculation.

Treatments
1) Brimonidine tartrate 0.05% aerosol, generated with ultrasonic nebulizer (12 ml solution loaded into nebulizer for each treatment), delivered into a holding chamber, and breathed spontaneously by awake rats for 5 minutes twice daily (0800 and 1800 hrs), beginning one day after viral inoculation.
2) Dexmedetomidine HCl 0.05% aerosol, generated with ultrasonic nebulizer (12 ml solution loaded into nebulizer for each treatment), delivered into a holding chamber, and breathed spontaneously by awake rats for 5 minutes twice daily (0800 and 1800 hrs), beginning one day after viral inoculation.
3) Control treatment: pH-matched saline aerosol.

A 5-minute exposure is recommended due to the lag time of filling the exposure box with aerosol after the rats have been loaded into the box

### Viral Infection

Rats will be inoculated with Parainfluenza type 1 (Sendai) virus via aerosol exposure, and housed in isolation cubicles. Control groups will be sham-inoculated with virus-free vehicle, and housed in an identical manner.

### Assessment

daily body weights;
lung function: oxygenation on room air (pulse oximetry); lung mechanics (pressure-volume curve, quasistatic elastance, dynamic elastance); airflow resistance (Newtonian resistance, respiratory system resistance, tissue damping);
lung inflammation: right lung bronchoalveolar lavage, with total leukocyte and differential leukocyte counts;
pulmonary transudate & exudates: left lung wet/dry weight ratio determined for 6 rats in each group;
formalin-fixed, paraffin-imbedded left lungs from 2 rats in each group; mid-sagittal thin sections prepared with H&E stain.

### Example 4 (Unpublished Data)

The purpose of this experiment was to test the effect of 0.05% brimonidine on mice in a lipopolysaccharide sepsis (LPS) model.

### Experimental Design

0.15% brimonidine solution was diluted to 0.05% brimonidine with 0.5% CMC. The animals were divided into three groups of eight mice each: 1) brimonidine-treated group; 2) control group (untreated mice with induced LPS sepsis); and 3) steroid-treated group.

In all three groups, LPS-induced sepsis was caused by delivery of highly concentrated LPS (50 μg) at 24 hour. In the brimonidine-treated mice, brimonidine was delivered for 5 minutes through intranasal drip pulmonary delivery at 0 hr, 6 hr, and 23.5 hr. In the steroid-treated mice, an inhaled corticosteroid (ICS) was delivered for 5 minutes through intranasal drip pulmonary delivery at 0 hr, 6 hr, and 23.5 hr. In the control group, no drug was administered prior to inducing LPS sepsis.

### Results

In the brimonidine-treated group, no animals died because of LPS-induced sepsis. In the control group, three out of eight animals died. In the steroid-treated group, no animals died. These results suggest that the inhaled brimonidine at low concentrations has anti-sepsis effects and has a potential to be used for the treatment of pulmonary diseases.

### Example 5 (Prophetic)

It is desired to develop a dexmedetomidine-based asthma drug to be used for preventive application. The drug will contain either dexmedetomidine by itself, ketamine by itself, or a combination of dexmedetomidine and ketamine. It is also desired to maximize the local diffusion and depot slow release, and minimize the peak plasma drug levels.

### Proposed Testing

For each candidate drug, the pKa is determined. Then, the pH is adjusted to be at least 0.5 units more basic. For example, pKa of dexmedetomidine is 7.1 and pKa of ketamine is 7.5. Accordingly, pH values of between 7.8 and 8.5 are preferred.

To demonstrate the advantages of the proposed pH range, one can compare the estimated local and systemic effects of dexmedetomidine at pH of 5.0 and pH of 8.0.

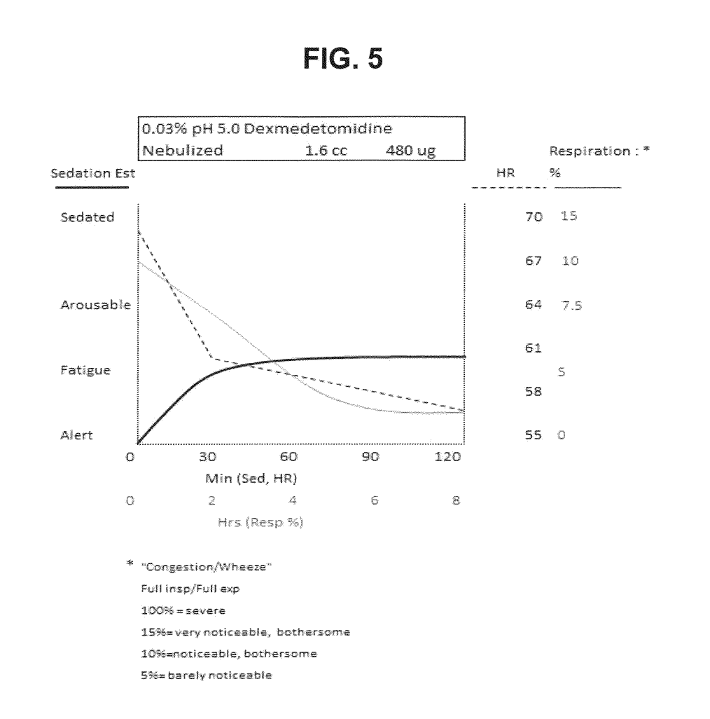

FIG. 5 depicts a chart that shows the prophetic estimation of local versus systemic effects of nebulized 0.03% dexmedetomidine at pH 5.0, 1.6 cc, 480 μg.

FIG. 5 demonstrates that there is an anticipated sedation (solid black line) over the first 20 minutes when peak plasma levels are approached, after which the sedation levels off. The increasing sedation is mild but noticeable, rendering the drug effective for the treatment of acute attacks but likely rendering it unsuitable as a routine preventative that can be used during waking hours while performing normal activities. Also, there is a substantial bradycardia (dashed line) for the α-2 agonist or tachycardia if ketamine were employed at this pH. There is also a noticeable positive respiratory effect (light gray line) as demonstrated in a reduced “wheeze-auscultation” score from 10 to about 5 on a scale for mild asthma, such as may occur in exercise induced asthma. An individual with a more substantial asthma than this baseline may demonstrate much more substantial effect.

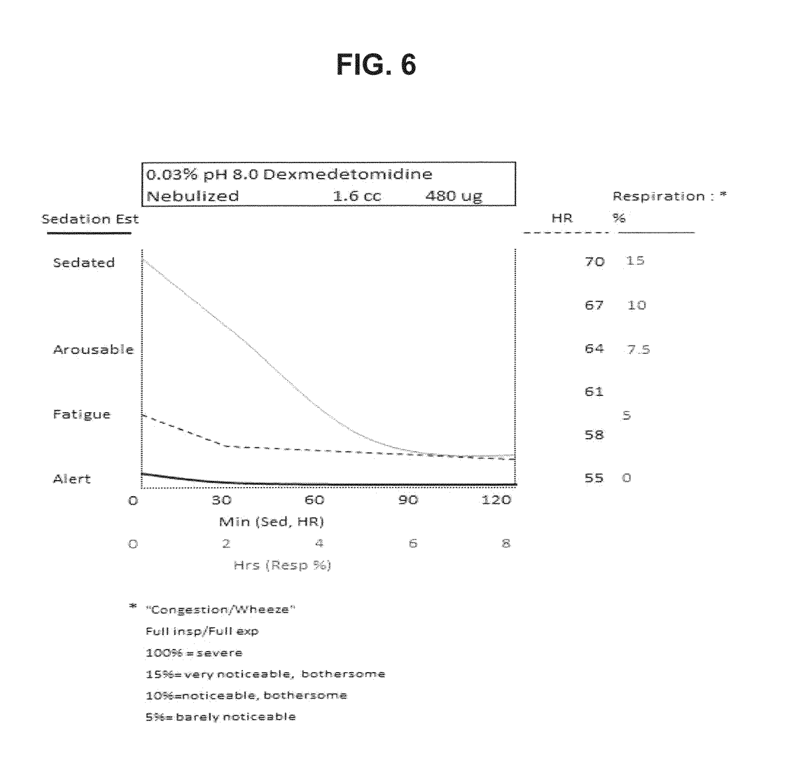

FIG. 6 depicts a chart that shows the prophetic estimation of local versus systemic effects of nebulized 0.03% dexmedetomidine at pH 8.0, 1.6 cc, 480 μg. In contrast with the same drug at pH 5.0, the effect of the drug on the heart rate is much reduced. There is virtually no sedation. Further, the lung effects are greater, with a more substantial reduction in wheeze and airway resistance versus baseline, which in this case was shown as a mild condition.

The doses are estimates only, assuming less than 20% nebulized DTL (dose to lung).

What is claimed is:

1. A composition comprising a selective α-2 adrenergic receptor agonist having a binding affinity of 300 fold or greater for α-2 over α-1 adrenergic receptors, or a pharmaceutically acceptable salt thereof, wherein pH of said composition is between about 4.0 and about 8.5.

2. The composition of claim 1, wherein said α-2 adrenergic receptor agonist is present at a concentration from between about 0.001% to about 0.1% weight by volume.

3. The composition of claim 1 wherein the total amount of the selective α-2 agonist is between about 10 μg and about 1000 ug.

4. The composition of claim 1, wherein said selective α-2 adrenergic receptor agonist has a binding affinity of 700 fold or greater for α-2 over α-1 adrenergic receptors.

5. The composition of claim 1, wherein said selective α-2 adrenergic receptor has a binding affinity of 100 fold or greater for α-2b and/or α-2c receptors over α-2a adrenergic receptors

6. The composition of claim 1, wherein said selective α-2 adrenergic receptor agonist is selected from the group consisting of brimonidine, dexmedetomidine, guanfacine, guanabenz, clonidine, and mixtures of these compounds.

7. The composition of claim 1 wherein said α-2 agonist has a Log P value of between about 2.0 and about 4.5.

8. The composition of claim 1, wherein said composition further comprises magnesium chloride or magnesium sulfate.

9. The composition of claim 1, wherein said composition further comprises sodium citrate.

10. A composition comprising between about 0.01% to about 0.05% weight by volume of a selective α-2 adrenergic receptor agonist having a binding affinity of 1000 fold or greater for α-2 over α-1 adrenergic receptors, or a pharmaceutically acceptable salt thereof, and wherein pH of said composition is between about 4.0 and about 8.5.

11. The composition of claim 10 wherein the pH is between about 6.5 and about 8.5.

12. The composition of claim 1, wherein said selective α-2 adrenergic receptor agonist is brimonidine.

13. The composition of claim 1, wherein said selective α-2 adrenergic receptor agonist is dexmedetomidine.

14. The composition of claim 1, wherein said composition is aerosolized.

15. The composition of claim 1, further comprising a bronchodilator.

16. The composition of claim 15, wherein said bronchodilator is selected from the group consisting of β-2 adrenergic receptor agonists, anticholinergics, and theophylline.

17. The composition of claim 1, further comprising ketamine.

18. A composition comprising between about 0.01% to about 0.05% weight by volume of dexmedetomidine and a pharmaceutically effective amount of ketamine for the treatment of a pulmonary disease or condition, wherein pH of said composition is between about 7.6 and about 8.2.

19. A composition comprising a pharmaceutically effective amount of ketamine for the treatment of a pulmonary disease or condition, wherein pH of said composition is between about 7.0 and about 8.5.

20. The composition of claim 18, wherein the pH is between about 7.8 and about 8.5.

21. The composition of claim 18, wherein said ketamine comprises S-enantiomer of ketamine.

22. The composition of claim 18, wherein the amount of ketamine is between about 5 μg and about 500 μg.

23. A method of treating a pulmonary disease or condition in a patient in need thereof comprising administering to said patient a pharmaceutically effective amount of the composition of claim 1.

24. The method of claim 23, wherein said pulmonary disease or condition is selected from the group consisting of asthma, pneumonia, edema, respiratory syncytial virus (RSV) disease, viral upper respiratory tract infection (URI), lower respiratory tract infection (LRI), cystic fibrosis, acute respiratory distress syndrome, bronchiolitis, and acute lung injury.

25. The method of claim 23, wherein said pulmonary disease or condition comprises viral upper respiratory tract infection (URI) and/or lower respiratory tract infection (LRI).

26. A method of treating a pulmonary disease or condition in a patient in need thereof comprising administering to said patient a pharmaceutically effective amount of the composition of claim 18.

27. The method of claim 26, wherein said ketamine is nebulized at about 12.5 to 25 μg/ml and is administered to said patient for about 5 to 20 minutes.

28. The method of claim 26, wherein said ketamine is nebulized at about 0.10% and is administered to said patient for about 5 to 10 minutes.

29. The method of claim 23 wherein said pulmonary disease or condition comprises asthma.
